(19) United States
(12) Patent Application Publication
(10) Pub. No.: US 2018/0036511 A1
(43) Pub. Date: Feb. 8, 2018

(54) IMPROVED CATHETER HANDLE

(71) Applicant: CathRx Ltd, Homebush Bay, NSW (AU)

(72) Inventors: Garrett Ryan, Surry Hills (AU); David Ogle, Cowan (AU); Roman Greifeneder, Bexley (AU)

(21) Appl. No.: 15/550,651

(22) PCT Filed: Jan. 12, 2016
(86) PCT No.: PCT/AU2016/050007

§ 371 Date: Aug. 11, 2017

(30) Foreign Application Priority Data
Feb. 13, 2015 (AU) 2015900471
Feb. 26, 2015 (AU) 2015900675

(51) Int. Cl. A61M 25/01

(52) U.S. Cl. CPC A61M 25/0136; A61M 25/0147; A61M 25/0662

(57) ABSTRACT

A catheter handle assembly comprising a handle body with a proximal end and a distal end with a passage extending therebetween. An adjustment unit disposed in the passage of the handle body and the adjustment unit comprising a gear mechanism that acts on the on a deflection stylet for adjusting deflection of the distal part of the deflection stylet. The gear mechanism comprises a first rack, a second rack and a pinion such that rotation of the pinion causes relative movement of the first rack and the second rack in opposing directions.

### CROSS-REFERENCE TO RELATED APPLICATIONS

This application is a national phase entry under 35 U.S.C. § 371 of International Patent Application PCT/AU2016/050007, filed Jan. 12, 2016, designating the United States of America and published in English as International Patent Publication WO 2016/127203 A1 on Aug. 18, 2016, which claims the benefit under Article 8 of the Patent Cooperation Treaty to Australian Patent Application Serial No. 2015900675, filed Feb. 26, 2015, and to Australian Patent Application Serial No. 2015900471, filed Feb. 13, 2015.

### TECHNICAL FIELD

The present invention generally relates to a gear mechanism assembly and, more particularly, to a catheter handle assembly including the gear mechanism assembly.

### BACKGROUND

In the field of cardiac procedures, use is made of a catheter that is steered through a patient's vasculature and placed at the desired site. Generally, the site is within a heart of the patient and a distal part of the catheter needs to be maneuvered into position against heart wall tissue. To be able to manipulate the distal part of the catheter to place it in tissue contact, the end of the catheter is flexible and steerable. A deflectable stylet is associated with the catheter for deflecting the distal part of the catheter.

Often, the part of the heart wall that needs to be accessed is awkwardly situated resulting in it being difficult to place the distal part of the catheter in contact with the desired part of the tissue to be treated or diagnosed.

Known catheters usually use a pull-wire arrangement in order to manipulate a stylet within a catheter sheath. These wire arrangements can use a 1:1 ratio pulley system to effect manipulate the end of a catheter sheath. Some known catheters include a gear mechanism comprising a rack and pinion mechanisms housed within a catheter handle. Actuation of these gear mechanisms usually effects the deflection of the distal end of a catheter to track along a tortuous anatomy. However, these single rack mechanisms usually require a significant amount of force to manipulate and may cause unnecessary strain on a clinician or physician when using the device over multiple procedures/multiple deflection cycles. It would be advantageous for a catheter to include a gear mechanism that reduced unnecessary strain/force required to apply a deflection. It would be further advantageous to have a higher gear ratio to effect manipulation.

Further, while not commonly used, these rack and pinion gear mechanisms usually take up a significant amount of internal space within the catheter handle. As such, it would be advantageous for a gear mechanism to be of a compact size. Further, it would be beneficial to manufacture a gear mechanism that is of a low manufacturing cost.

Often, the use of a catheter can impart a great stress for a clinician or physician due to the strain during manipulation of the catheter. It would be advantageous to have a catheter in which the stress and/or strain felt by the clinician or physician could be reduced.

Other gear mechanisms including a rack and pinion have been known to malfunction or have the pinion come off of the rack under relatively large amounts of force. It would be advantageous for a gear mechanism that resisted separation of the pinion from the rack under relatively large amounts of force.

Any discussion of the prior art throughout the specification should in no way be considered as an admission that such prior art is widely known or forms part of common general knowledge in the field.

### BRIEF SUMMARY

It may be an object of the present disclosure to provide an improved gearing mechanism adapted for use with catheters.

It is an object of the present disclosure to overcome or ameliorate at least one of the disadvantages of the prior art, or to provide a useful alternative.

A first aspect of the present disclosure may relate to a catheter handle assembly comprising; a handle body having a proximal end and a distal end with a passage extending therebetween; an adjustment unit disposed in the passage of the handle body, the adjustment unit comprising a gear mechanism that acts on a deflection stylet for adjusting deflection of the distal part of the stylet; and wherein the gear mechanism comprises a first rack, a second rack and a pinion such that rotation of the pinion causes relative movement of the first rack and the second rack in opposing directions.

The catheter handle may further comprise a carrier arrangement projecting from the distal end of the handle body, the carrier arrangement configured for mounting a catheter sheath and at least a part of a deflection stylet of a catheter thereto during use of the catheter handle assembly.

The second rack may be stationary relative to the catheter handle. Further, the adjustment unit may have a gear ratio of at least 1.0:1.1. Wherein the adjustment unit may have a gear ratio of 1.0:2.0. Wherein the adjustment unit may have a gear ratio of 1.0:3.0. Wherein the first rack and the second rack may be facing the stylet holder when the catheter is not in use. Wherein a shell may be disposed around an axis of the gear mechanism to retain the pinion on at least one of the first rack and the second rack. The second rack may be disposed on the shell of the gear mechanism. The shell may comprise an inner shell portion and an outer shell portion. the first rack may be disposed on a stylet mount. The stylet mount may engage the catheter sheath and the stylet holder may engage the stylet such that movement of the pinion may cause a relative movement between the stylet and the catheter sheath.

In a further aspect of the present disclosure there is provided a gear mechanism for a catheter handle assembly, the gear mechanism comprising; a first rack disposed on a stylet mount; a second rack disposed on a portion of a shell wherein the rotation of a pinion causes opposing relative movement of the first rack and the second rack in the catheter handle.

In yet another aspect of the present disclosure there is provided a gear mechanism comprising at least one novel and inventive feature as disclosed in the present specification.

In another aspect of the present disclosure there is provided a catheter handle comprising a gear mechanism with at least one novel and inventive feature as disclosed in the specification.

In at least one embodiment, the frictional force applied by the gear mechanism can be altered via a friction-inducing device such as a tapered pinion shaft. Wherein the friction can be tuned by the user to suit the desired range.

In the context of the present invention, the words “comprise,” “comprising” and the like are to be construed in their inclusive, as opposed to their exclusive, sense, that is in the sense of “including, but not limited to.”

The invention is to be interpreted with reference to the at least one of the technical problems described or affiliated with the background art. The present disclosure aims to solve or ameliorate at least one of the technical problems and this may result in one or more advantageous effects as defined by this specification and described in detail with reference to the preferred embodiments of the present invention.

### BRIEF DESCRIPTION OF THE DRAWINGS

FIG. 1 shows a perspective view of an embodiment of a catheter assembly;

FIG. 2 shows a partial cutaway view of the catheter assembly;

FIG. 3 shows an exploded view of a gear mechanism of the catheter assembly;

FIG. 4A shows a perspective view of the gear mechanism of the catheter assembly;

FIG. 4B shows a front view of an embodiment of the gear mechanism of the catheter assembly;

FIG. 4C shows a side view of an embodiment of the gear mechanism of the catheter assembly;

FIG. 5A shows a further embodiment of the gear mechanism of the catheter assembly;

FIG. 5B shows a front view of a further embodiment of the gear mechanism of the catheter assembly;

FIG. 5C shows a side view of a further embodiment of the gear mechanism of the catheter assembly;

FIG. 6 shows a perspective view of another embodiment of a catheter assembly with two control knobs.

### DETAILED DESCRIPTION

Preferred embodiments of the present disclosure will now be described with reference to the accompanying drawings and non-limiting examples.

In the drawings, reference numeral 10 generally designates a preferred embodiment of a catheter assembly 10. The catheter assembly 10 includes a handle assembly 12. A catheter sheath 14 extends from a distal part 16 of the handle assembly 12. The catheter sheath 14 defines a lumen, which is a deflection stylet lumen for receiving a deflection stylet.

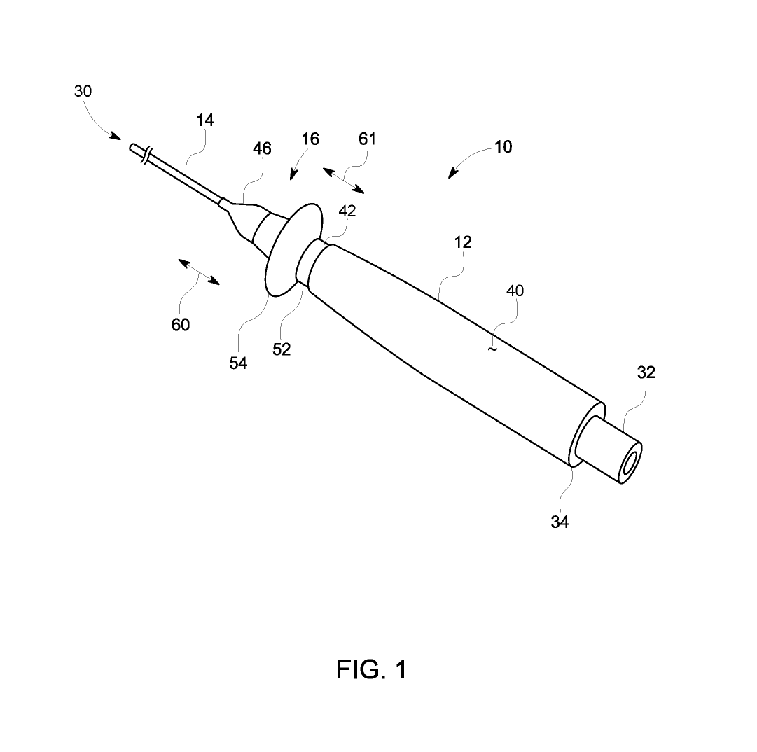

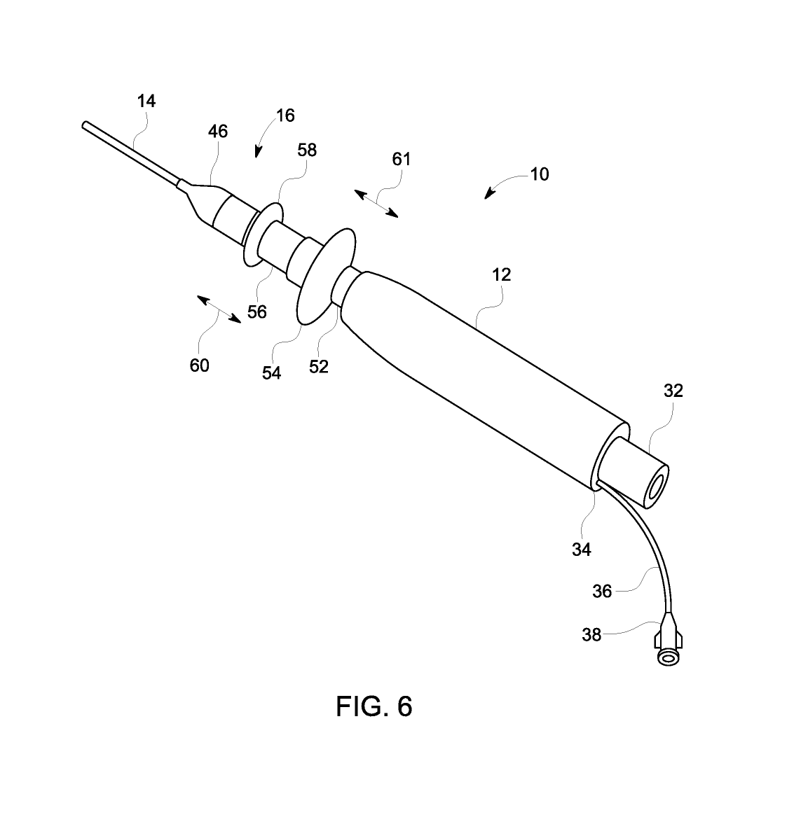

In a further embodiment, the catheter sheath 14 defines a plurality of lumens. One of the lumens is a conductor lumen and has a plurality of conductors (not shown (including thermocouple conductors)) received therein. The plurality of conductors extend from electrodes carried on a distal part (not shown) of the catheter sheath 14 toward a proximal end of the catheter sheath 14. The plurality of conductors further extend through the handle assembly 12 to an electrical connector 32 (FIG. 1) arranged at a proximal end 34 of the handle assembly 12. The catheter sheath 14 is releasably connected to the distal part 16 of the handle assembly 12. The catheter sheath 14 and the handle assembly 12 each comprise suitable connectors (not shown in detail) for connecting the plurality of conductors from the catheter sheath 14 to the distal end 16 of the catheter handle assembly 12. The stylet is arranged to fit through the connectors of the catheter sheath 14 and the distal end 16 of the handle into the deflection stylet lumen. One of the lumens is an irrigation lumen for providing irrigating fluid to the electrodes at a distal part 30 of the catheter sheath 14. This lumen communicates with a fluid conduit 36 (FIG. 6). A luer connector 38 is arranged at a proximal end of the conduit for connection to a supply of irrigation fluid (not shown).

An embodiment of the handle assembly 12 is now described in greater detail with reference to FIG. 1 of the drawings.

The handle assembly 12 includes a tubular handle body 40 defining the proximal end 34 to which the connector 32 (FIG. 1) is integrated and a distal end 42. A carrier arrangement projects from the distal end 42 of the tubular handle body 40. The carrier arrangement mounts the catheter sheath 14, via a strain relief unit 46, and at least a part of a proximal region of the deflection stylet. An adjustment unit is arranged in a passage of the handle body.

The carrier unit comprises a deflection unit in the form of a first tubular member or tube 52 having a control knob 54 arranged at a distal end of the tube 52. Optionally, the carrier arrangement further comprises a size selector unit in the form of a second tubular member or second tube 56. Once again, the second tube 56 carries a control knob 58 (see FIG. 6) at a distal end of the second tube 56. The tubes 52 and 56 are telescopically arranged with respect to each other and with respect to the distal end of the handle body. Thus, the tube 52 can be slid axially with respect to the catheter handle 12, and in the direction of arrows 60 and 61 (FIGS. 1 and 6) as will be described in greater detail below.

The adjustment unit comprises a gear mechanism 62 associated with the tube 52. The deflection stylet comprises a tubular member having a bend-enhancing portion (not shown) proximate its distal end. An actuator, which may be in the form of a wire, is received in the tubular member and a distal end of the actuator is fastened with a distal part of the tubular member, distally of the bend-enhancing portion of the tubular member. The bend-enhancing portion may comprise any suitable bend-enhancing region, which forms a zone of weakness at the distal part of the catheter to allow bending around the bend-enhancing portion. For example, the bend-enhancing portion could be groups of slots, a longitudinally extending scalloped region, or the like.

The stylet optionally includes a size selector in the form of a sleeve, which is displaceably arranged with respect to the tubular member of the stylet for interacting with the bend-enhancing portion to increase or reduce the size of the bend-enhancing portion and, in so doing, to control the degree of curvature of the distal part 30 of the catheter sheath 14 (in which the stylet is received).

The sleeve is mounted over the tubular member. It will, however, be appreciated that the sleeve could be received within the tubular member so that it is interposed between the tubular member and the actuator.

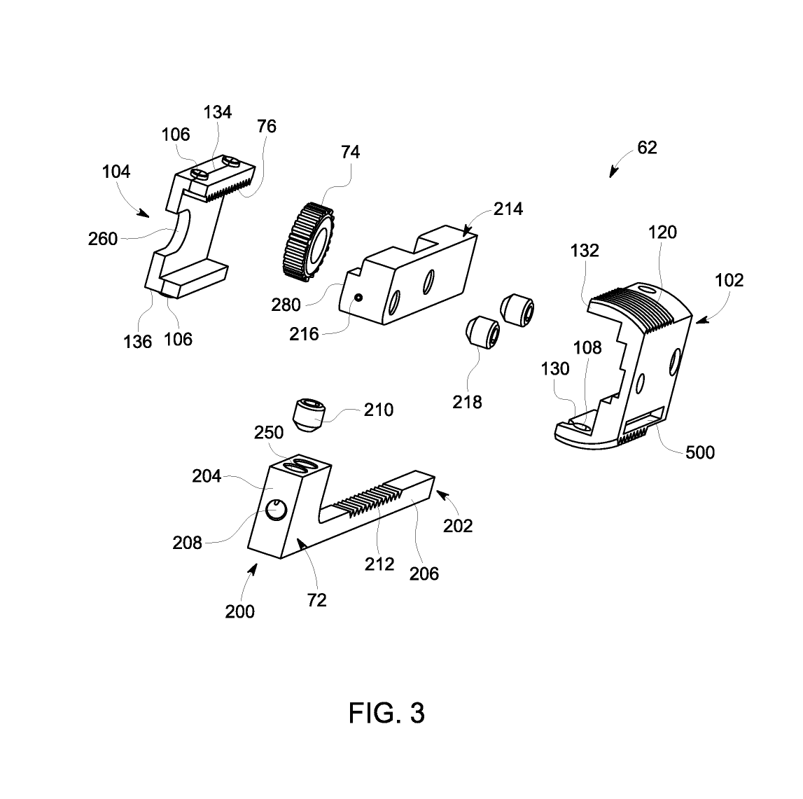

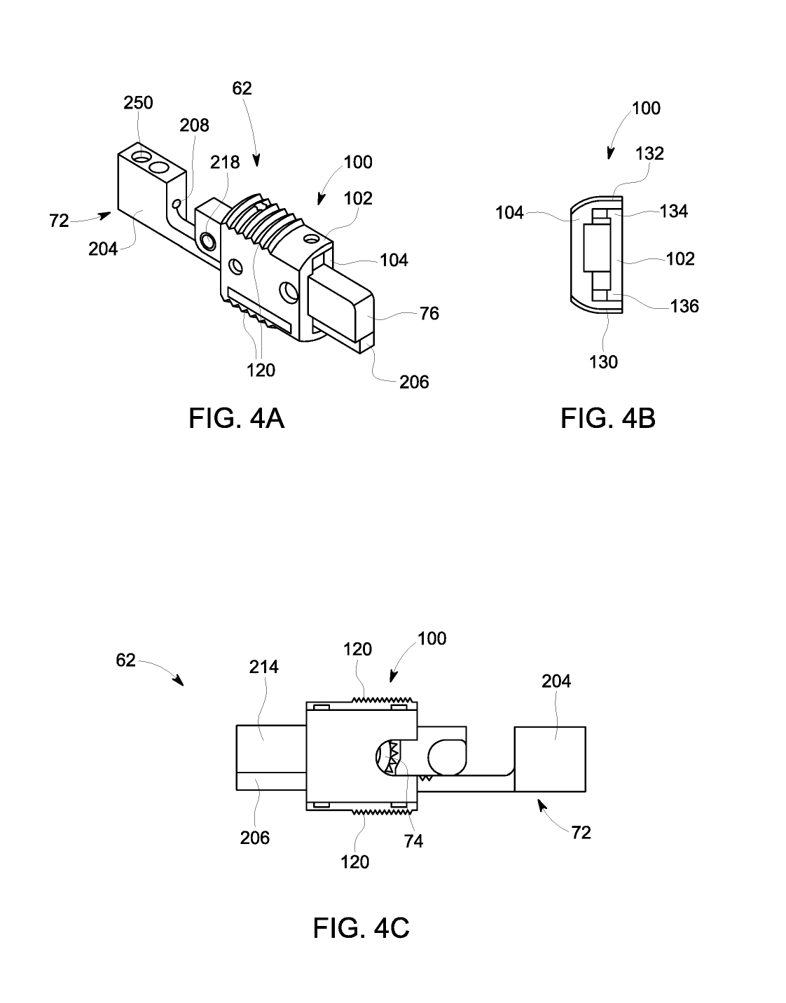

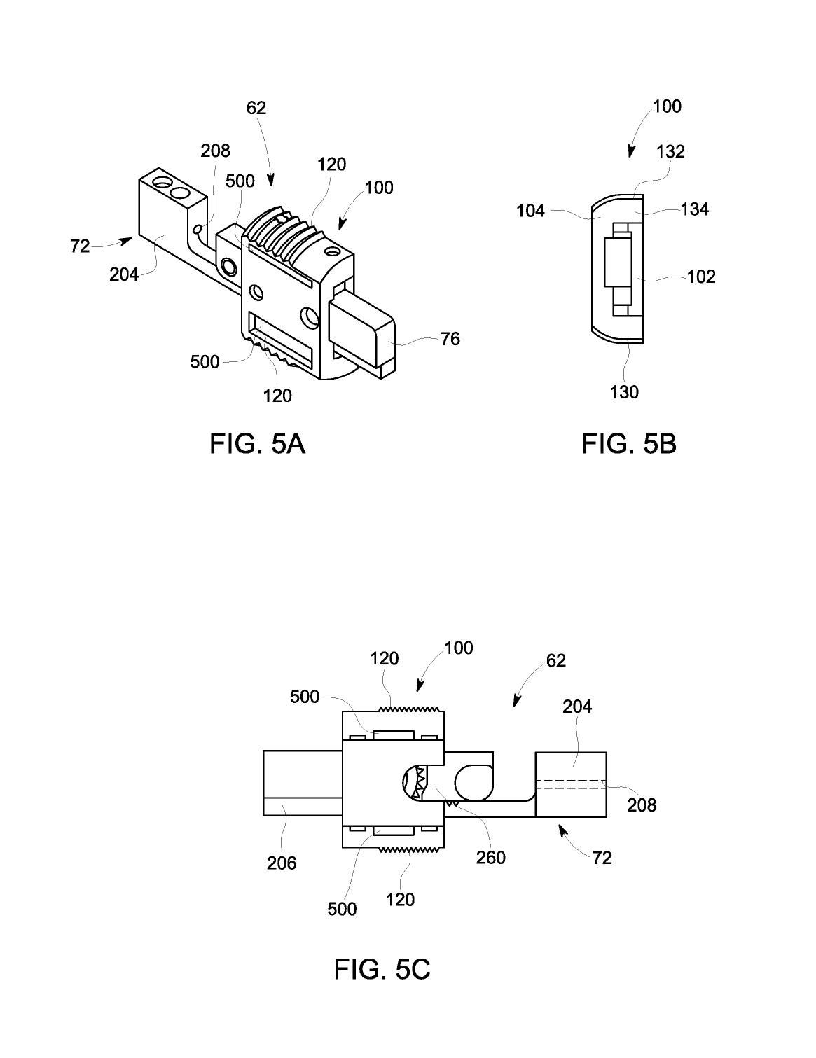

The gear mechanism 62 comprises a drive gear in the form of a stylet mount 72 (FIG. 3) extending proximally from a proximal end of the tube 52 of the carrier arrangement. The stylet mount 72 comprises a proximal end 200, a distal end 202, a head portion 204 and a longitudinal body portion 206 extending proximally from the head portion 204. The head portion 204 of the stylet mount 72 is adapted to receive the catheter sheath 14 in aperture 208. Referring to FIGS. 4A and 5A, head portion 204 can optionally be of a larger dimension compared to that of the longitudinal body portion 206 such that the catheter sheath 14 can be mounted in the aperture 208. In some embodiments, the larger dimension of the head portion 204 can be used to resist stresses applied to an adjustment unit 48 during use and can also be used as a strain relief for the catheter sheath 14.

In at least one embodiment, the catheter sheath 14 is anchored to the stylet mount 72 via grub screw 210 in aperture 250, but it will be appreciated that any suitable means can be used for securing the catheter sheath 14 to the stylet mount 72. The longitudinal body portion 206 of the stylet mount 72 comprises a first rack 212, which is in a cooperative relationship with a pinion 74 of a shuttle 214 for effecting relative movement between the stylet and the catheter sheath 14, and thus causing deflection of the distal end of the catheter sheath 14.

The pinion 74 is rotatably mounted to the shuttle 214, wherein the shuttle 214 is in the form of a stylet holder 214, which retains the stylet 24 in aperture 216. FIG. 3 illustrates a grub screw 218 for releasably retaining the stylet within the stylet holder 214, however it will be appreciated that other suitable means can be used to retain the stylet in the aperture 216, such as gluing. Pinion 74 is rotatably attached to the stylet holder 214 such that rotation of the pinion 74 causes relative movement between the pinion and at least the first rack 212. In at least one embodiment, a side of the stylet holder is in an abutting relationship with the stylet mount and wherein the abutting sides of the stylet holder and the stylet mount are flush or otherwise in a substantially corresponding surface relationship. The stylet holder can have a rounded proximal end illustrated in FIG. 4A. Further the stylet holder 214 can also have a protrusion 280 for abutting the head portion 204.

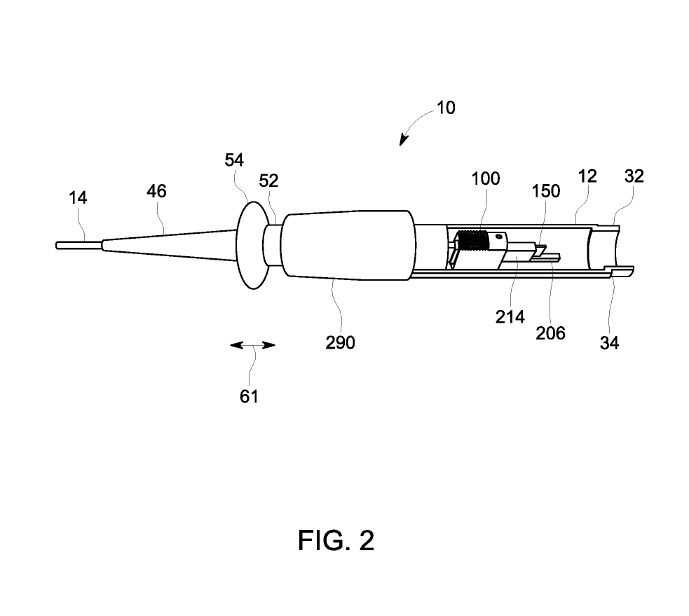

Shell 100 illustrated in FIG. 2 and FIG. 3 can be used to assist in retaining pinion 74 on the stylet mount 72. Further, shell 100 can isolate the gear mechanism 62 from other components in the passage of the catheter handle assembly 12 to reduce the possibility of damage to the other components within the passage. The shell 100 comprises an outer shell portion 102 and an inner shell portion 104. The inner shell portion 104 and the outer shell portion 102 can comprise at least one male component 106 and at least one female component 108, respectively, and operatively associated such that the outer shell portion 102 and inner shell portions 104 can be press-fit together to form shell 100. It will be appreciated that outer shell portion 102 and inner shell portion 104 can be connected by any other suitable means such as screws, tongue-in-groove, glue, press-fit lock, rail slide arrangements or the like.

The inner shell portion 104 further comprises a second rack 76 which is in a cooperative relationship with pinion 74. When the second tube 56 is withdrawn proximally fully into the tube 52, the pinion moves linearly along both the first rack 212 and the second rack 76 such that the pinion 74 effects relative movement between first rack 212 and the second rack. This relative movement is an opposing directional movement such that when the first rack 212 moves proximally relative to the pinion 74, the second rack 76 moves distally relative to the pinion 74. The gear ratio of the first rack 212 relative to the second rack can be in the range of 1.0:0.5 to 1.0:3.5. Preferably the gear ratio is greater than 1.0:1.0. More preferably, the gear ratio is in the range of 1.0:1.0 to 1.0:2.0. Even more preferably, the gear ratio is 1.0:1.5. The two shell portions 102, 104 form an aperture which receives at least a portion of the stylet mount, more particularly the longitudinal body portion, and the stylet holder 214. In this configuration the pinion 74 is rotatable such that it can impart movement to at least one of the first rack and the second rack.

In at least one embodiment, the shell 100 is fixed to the catheter handle such that the second rack 76 is stationary relative to the catheter handle assembly 12. The shell 100 can be removably attached or fixed to the catheter handle assembly 12 at connection portions 120 disposed on the outer surface of the shell 100. Alternatively the connection portions 120 can be used for providing a resistive force to another component within the catheter handle assembly 12 (not shown). A protrusion 150, illustrated in FIG. 2, is formed in the passage of the catheter handle assembly 12 to retain the gear mechanism 62 at the desired location in the passage. Preferably the connection portion 120 engages the interior wall of the catheter handle assembly 12 to secure the shell 100 in a desired location.

The outer shell portion 102 comprises two wing portions 130, 132 that can be used to retain the inner shell portion 104 at sides 134, 136. The inner shell portion 104 can have a cutaway portion 260 such that the gear mechanism 62 can be viewed to ensure alignment on the on the first and second racks 212, 76, respectively, or otherwise view the rotation of the pinion 74. The cutaway portion 260 can also be used to allow additional flexure of the inner portion 104 during connection of the outer shell portion 102 and the inner shell portion 104.

Referring to FIGS. 4A through 4C, there is illustrated an embodiment of the gear mechanism 62 of the present disclosure. The shell 100 of the gear mechanism 62 has a compact configuration to reduce the space occupied within the passage of the catheter handle.

The adjustment unit is of a compact size and allows for a reduction of forces during operation. More particularly, the adjustment unit can reduce the forces experienced by a user by up to around ⅔. More particularly, the reduction of forces experienced by a user is around half. This reduction of forces can also be experienced by the adjustment unit and gear mechanism in the passage of the catheter handle. Further, a friction inducer (not shown) allows the clinician or physician to adjust or tune the stickion-friction of the catheter assembly such that the friction of the catheter can be increased or decreased. Further, adjusting the stickion-friction of the catheter assembly can reduce wear and tear of the internal components.

In at least one embodiment, the frictional force applied by the gear mechanism can be altered via a friction inducing device (not shown) such as a tapered pinion shaft. This allows for greater ease of use of the catheter and reduces the stresses experienced by the physician during use.

FIGS. 5A to 5C illustrate an alternative embodiment of the gear mechanism 62 of the present disclosure. The shell 100 of the gear mechanism has an enlarged cross-section to extend to the sides of the passage of the catheter handle for larger catheter handles. This configuration can have at least one depression 500 on at least one outer surface of the shell 100 as seen in FIG. 5A.

The first rack 212 and the second rack 76 are facing the stylet holder 214 such that the pinion 74 is simultaneously operatively engaged with the first rack 212 and the second rack 76.

The catheter handle assembly 12 may also comprise a grip portion 290 (FIG. 2.) to reduce repetitive stress or user stress experienced by the clinician or physician using the catheter. The grip portion comprises a plurality of scalloped portions around the exterior of the catheter handle.

In at least one embodiment (FIG. 6), pinion 74 is rotatably received in the passage of the handle body and a stylet holder 214 arranged in the passage of the tubular handle body. The stylet holder 214 mounts a proximal end of the actuator (stylet). The tubular member of the stylet is anchored within the tubular handle body 40. Thus, by displacing the deflector unit of first tubular member 52 and control knob 54 in the direction of arrows 61 (FIG. 6), relative movement between the tubular member and the actuator occurs, resulting in deflection of the distal part of the catheter sheath 14. The distal part 30 can deflect in a direction by appropriate manipulation of the deflector unit of first tubular member 52 and control knob 54 in the opposite direction of the longitudinal axis of the catheter sheath 14 but within a plane. The in-plane deflection is enhanced by having the stylet lumen eccentrically arranged within the catheter sheath 14.

To facilitate the bi-directional deflection of the distal part of the catheter sheath 14, when the deflector unit of first tubular member 52 is in a rest position, i.e., the position in which the distal part 30 extends longitudinally, the pinion 74 is substantially centrally arranged on the racks 212 and 76, i.e., inwardly of ends of the racks 212 and 76. This allows push-pull movement of the actuator 68 in the direction of arrows 60, 61 (FIG. 6) so that bi-directional deflection occurs.

When the second tube 56 is withdrawn proximally fully into the tube 52, the first rack 212 drives the pinion 74 and second rack 76 to urge the sleeve into the stylet lumen of the catheter sheath 14 to occlude a part of the bend-enhancing portion of the stylet, resulting in a tighter radius of curvature. Conversely, when the second tube 56 is extended distally with respect to the tube 52, the first rack 212 drives the pinion 74 and second rack 76 to withdraw the sleeve from the stylet lumen, exposing more of the bend-enhancing portion of the stylet and allowing for a greater radius of curvature.

The handle body is made up of mating parts so that the parts can be separated to enable access to be gained to the passage of the handle body. The proximal end of the actuator is releasably attached to the stylet holder 214 and the tubular member of the stylet is, likewise, releasably mounted in the passage of the handle body. The releasable connection is preferably arranged by molding a slot or another similar receiving formation 216 on the stylet holder 214 where the proximal end of the actuator can be securely fitted. Likewise, the handle body comprises a slot or another suitable receiving formation molded in the passage for securely but releasably fitting the proximal end of the tubular member of the stylet into the handle body. The proximal end of the actuator and the proximal end of the tubular member each have a mounting formation (not shown in figures) such as an enlarged fitting at the respective proximal ends. The mounting formation is a snug fit in the corresponding receiving formation in the stylet holder 214 and the handle body. The sleeve is attached in a similar fashion to the stylet mount 72 having a suitable receiving formation 208 for the sleeve. One or more screws may be used to ensure that each of the actuator, tubular member, and sleeve are securely anchored while the catheter is in use.

In an alternative embodiment of the invention, the sleeve is connected directly to the size selector tube 56 so that the adjustment unit only comprises one gear mechanism 62 for deflecting the distal end of the catheter sheath 14. In this embodiment, displacing the size selector tube 56 and control knob 58 in the direction of arrows 60, 61 (FIG. 6) results in displacement of the sleeve directly within the catheter sheath 14.

In at least one embodiment, the first rack 212 and the second rack 76 are generally parallel with respect to one another and the respective racks of the first and second racks 212, 76 are facing towards the pinion. In this configuration, the rotation of the pinion 74 causes the first rack 212 to move in an opposing direction relative to that of the second rack 76. It will be appreciated in some embodiments that the second rack 76 is fixed to the catheter handle such that proximal movement of the pinion 74 causes a proximal movement of the stylet mount 72.

The handle body can be opened by separating the mating parts, the component parts of the deflection stylet disconnected from the adjustment unit and the deflection stylet removed. Similarly, the catheter sheath 14 is removably mounted to the distal end 16 of the handle assembly 12. Thus, the catheter sheath 14 can also be removed from the handle assembly 12. In this way, the component parts of the catheter assembly 10, comprising the handle assembly 12, the deflection stylet and the catheter sheath 14 can be removed for reprocessing and/or replacement. It is envisaged that, in general, the handle assembly 12 and the deflection stylet can be reprocessed between twenty and fifty times, whereas the catheter sheath 14 can be reprocessed for approximately five re-uses. In this regard, the term “reprocessing” (and derivatives) is to be understood in a broad sense to include reprocessing, remanufacturing, refurbishment, or the like.

It will be appreciated that the gear mechanism of the present disclosure can be used with any catheter. In at least some embodiments at least one gear mechanism can be used to effect at least one of deflection of a stylet, impart a shape to a stylet, or otherwise manipulate a stylet in a predetermined manner. It will further be appreciated that more than one gear mechanism can be used within a catheter assembly.

Hence, it is an advantage of the disclosed embodiments that a modular catheter assembly 10 is shown that lends itself to reprocessing. It will be appreciated by those skilled in the art that using a catheter only once is a very expensive procedure as the costs of production of the catheter are significant. By reprocessing the catheter and using its constituent parts a number of times, a substantial reduction in costs for the institution and, as an end result, for the patient using the catheters, can be achieved.

It is a further advantage of the disclosed embodiments that a handle assembly 12 is disclosed having positive drive, i.e., the gear arrangement, for effecting deflection and size selection. Using a positive drive results in more accurate control over deflection and size selection and improved tactility for the clinician.

Reference throughout this specification to “one embodiment,” “some embodiments” or “an embodiment” means that a particular feature, structure or characteristic described in connection with the embodiment is included in at least one embodiment of the present invention. Thus, appearances of the phrases “in one embodiment,” “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified, the use of ordinal adjectives “first,” “second,” “third,” etc., to describe a common object, merely indicate that different instances of like objects are referred to, and are not intended to imply that the objects so described must be in a given sequence, either temporally, spatially, in ranking, or in any other manner.

In the appended claims and the description herein, any one of the terms “comprising,” “comprised of,” or “which comprises” is an open term that means including at least the elements/features that follow, but not excluding others. Thus, the term “comprising,” when used in the claims, should not be interpreted as being limitative to the means or elements or steps listed thereafter. For example, the scope of the expression “a device comprising A and B” should not be limited to devices consisting only of elements A and B. Any one of the terms “including,” “which includes,” or “that includes,” as used herein, is also an open term that also means including at least the elements/features that follow the term, but not excluding others. Thus, “including” is synonymous with and means “comprising.”

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some, but not other, features included in other embodiments, combinations of features of different embodiments are meant to be within the scope of the invention, and form different embodiments, as would be understood by those skilled in the art. For example, in the appended claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term “coupled,” when used in the claims, should not be interpreted as being limited to direct connections only. The terms “coupled” and “connected,” along with their derivatives, may be used. It should be understood that these terms are not intended as synonyms for each other. Thus, the scope of the expression “a device A coupled to a device B” should not be limited to devices or systems wherein an output of device A is directly connected to an input of device B. It means that there exists a path between an output of A and an input of B, which may be a path including other devices or means. “Coupled” may mean that two or more elements are either in direct physical or electrical contact, or that two or more elements are not in direct contact with each other but yet still cooperate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the invention.

It will be appreciated by persons skilled in the art that numerous variations and/or modifications may be made to the disclosure as shown in the specific embodiments without departing from the scope of the disclosure as broadly described. The present embodiments are, therefore, to be considered in all respects as illustrative and not restrictive.

Although the invention has been described with reference to specific examples, it will be appreciated by those skilled in the art that the invention may be embodied in many other forms, in keeping with the broad principles and the spirit of the invention described herein. The present invention and the described preferred embodiments specifically include at least one feature that is industrial applicable.

1. A catheter handle assembly comprising;
a handle body having a proximal end and a distal end with a passage extending therebetween;
an adjustment unit disposed in the passage of the handle body, the adjustment unit comprising a gear mechanism that acts on a stylet of a sheath for adjusting deflection of a distal part of the sheath; and
wherein the gear mechanism comprises a first rack, a second rack, a stylet holder and a pinion,
wherein the stylet holder is adapted to retain a stylet and has the pinion mounted thereon,
such that the pinion meshes the first rack and second rack, and
the rotation of the pinion causes relative movement of the first rack and the second rack in opposing directions.

2. The assembly of claim 1, wherein the second rack is stationary relative to the catheter handle.

3. The assembly of claim 1, wherein the adjustment unit has a gear ratio of at least 1.0:1.1.

4. The assembly of claim 3, wherein the adjustment unit has a gear ratio of 1.0:2.0.

5. The assembly of claim 3, wherein the adjustment unit has a gear ratio of 1.0:3.0.

6. The assembly of claim 1, wherein the first rack and the second rack are facing the stylet holder when the catheter is not in use.

7. The assembly of claim 1, wherein a shell is disposed around an axis of the gear mechanism to retain the pinion on at least one of the first rack and the second rack.

8. The assembly of claim 7, wherein the shell is disposed inside the handle body, wherein the handle body comprises mating parts.

9. The assembly of claim 6, wherein the second rack is disposed on the shell of the gear mechanism.

10. The assembly of claim 7, wherein the shell comprises an inner shell portion and an outer shell portion.

11. The assembly of claim 1, wherein the first rack is disposed on a stylet mount.

12. The assembly of claim 11, wherein the stylet mount is adapted to have a sleeve of the sheath mounted thereon.

13. The assembly of claim 11, wherein the stylet mount engages the sleeve and the stylet holder engages the stylet such that movement of the pinion causes a relative movement between the stylet and the catheter sheath.

14. The assembly of claim 1, further comprising a carrier arrangement projecting from the distal end of the handle body, the carrier arrangement configured for mounting a catheter sheath and at least a part of a deflection stylet of a catheter thereto during use of the catheter handle assembly.

15. The assembly of claim 1, wherein the gear mechanism comprises a friction inducing device such as a tapered pinion shaft for adjusting frictional force applied on the pinion.

16. A gear mechanism for a catheter handle assembly, the gear mechanism comprising:
a first rack, a second rack, a stylet holder and a pinion,
wherein the stylet holder is adapted to retain a stylet and has the pinion mounted thereon,
such that the pinion meshes the first rack and second rack, and
the rotation of the pinion causes relative movement of the first rack and the second rack in opposing directions.

17. The gear mechanism of claim 16, further comprising a friction inducing device for adjusting frictional force applied on the pinion.

18. The gear mechanism of claim 16, wherein, the friction can be tuned by the user to suit the desired range.

19. The gear mechanism of claim 17, wherein the friction inducing device comprises a tapered pinion shaft.
